(19) United States
(12) Patent Application Publication
(10) Pub. No.: US 2026/0325109 A1
(43) Pub. Date: Oct. 22, 2026

(54) ULTRASONIC WAVE DIAGNOSTIC DEVICE, METHOD FOR EXTRACTING TARGET RESULT, AND PROGRAM FOR EXTRACTING TARGET RESULT

(71) Applicant: Konica Minolta, Inc., Chiyoda-ku, Tokyo (JP)

(72) Inventor: Keita YOSHIDA, Hyogo (JP)

(73) Assignee: Konica Minolta, Inc., Chiyoda-ku, Tokyo (JP)

(21) Appl. No.: 19/491,107

(22) PCT Filed: Feb. 29, 2024
(86) PCT No.: PCT/JP2024/007641

§ 371 Date: Dec. 8, 2025

(30) Foreign Application Priority Data
Jun. 9, 2023 (JP) 2023-095504

(51) Int. Cl. A61B 8/00; G01S 7/52; G01S 15/89

(52) U.S. Cl. CPC A61B 8/5223; A61B 8/5207; A61B 8/523; G01S 7/52057; G01S 15/8934

(57) ABSTRACT

An ultrasonic wave diagnostic device includes: an image generation unit that generates, on the basis of a reflection signal of an ultrasonic wave outputted from a probe, cross-sectional images at different cross-sectional positions for a site of interest of a test subject; a measurement unit that measures to-be-measured values relating to the site of interest for each of the plurality of generated cross-sectional images; a target to-be-measured value extraction unit that extracts a target to-be-measured value which serves as a measurement target, on the basis of the measurement results of the plurality of to-be-measured values; and a target cross-sectional image extraction unit that extracts a target cross-sectional image corresponding to the target cross-sectional position, on the basis of the evaluation results of the plurality of cross-sectional images.

### TECHNICAL FIELD

The present invention relates to an ultrasonic (ultrasound) diagnostic apparatus, a target result extraction method, and a target result extraction program.

### BACKGROUND ART

An ultrasonic diagnostic apparatus is configured to, for example, generate a cross-sectional image (for example, a B-mode image) of a target part of a subject and display the generated cross-sectional image on a display of the ultrasonic diagnostic apparatus. A user, such as a medical doctor, refers to the cross-sectional image displayed on the display to perform diagnosis of the target part or perform measurement related to the target part.

In order to perform measurement related to a target part, for example, in Patent Literature (hereinafter referred as “PTL”) 1, it is evaluated whether a generated cross-sectional image is suitable for the measurement of the target part, and the measurement of the target part is performed using a cross-sectional image evaluated as being suitable for the measurement of the target part.

### CITATION LIST

### Patent Literatures

### PTL 1

### Japanese Patent Application Laid-Open No. 2020-137974

### SUMMARY OF INVENTION

### Technical Problem

In an ultrasonic diagnostic apparatus, for example, in a case where the crown rump length (hereinafter referred to as CRL) of a fetus is measured, the user applies an ultrasonic probe to a pregnant woman as a subject and performs an abdominal or transvaginal scan.

For the measurement of the CRL or the like, accuracy and efficiency are required. In this regard, in a case where the measurement is performed manually or semi-automatically, it has been necessary for the user himself/herself to select a cross-sectional image to be measured and to determine whether a measurement value measured by the user himself/herself is a target measurement value. For this reason, the accuracy is low, it takes time and effort, and the efficiency is low as well.

In PTL 1, it is evaluated on the side of the ultrasonic diagnostic apparatus whether a generated cross-sectional image is suitable for the measurement of a target part, and the target part is measured using the cross-sectional image evaluated as being suitable for the measurement as described above. In PTL 1, however, it is only evaluated whether a generated cross-sectional image is suitable for the measurement of a target part, and it is not determined whether the measured measurement value is the target measurement value, which cannot necessarily be said to be accurate. In addition, there is a case where a plurality of cross-sectional images is evaluated to be suitable for the measurement of a target part. Even this case cannot necessarily be said to be accurate, either, since it is not determined whether the measured measurement value is the target measurement value.

An object of the present invention is to provide an ultrasonic diagnostic apparatus, a target result extraction method, and a target result extraction program each capable of extracting an optimal measurement value and an optimal cross-sectional image.

### Solution to Problem

An ultrasonic diagnostic apparatus according to the present invention includes: an image generator that generates a plurality of cross-sectional images at different cross-sectional positions for a target part of a subject based on a reflection signal of an ultrasonic wave output from a probe; a measurer that measures a measurement value related to the target part for each of the plurality of generated cross-sectional images; a target measurement value extractor that extracts a target measurement value as a measurement target based on a measurement result of a plurality of the measurement values; and a target cross-sectional image extractor that extracts a target cross-sectional image corresponding to a target cross-sectional position based on evaluation results of the plurality of cross-sectional images.

A target result extraction method according to the present invention includes: generating a plurality of cross-sectional images at different cross-sectional positions for a target part of a subject based on a reflection signal of an ultrasonic wave output from a probe; measuring a measurement value related to the target part for each of the plurality of generated cross-sectional images; extracting a target measurement value as a measurement target based on a measurement result of a plurality of the measurement values; and extracting a target cross-sectional image corresponding to a target cross-sectional position based on evaluation results of the plurality of cross-sectional images.

A target result extraction program according to the present invention causes a computer to execute: generating a plurality of cross-sectional images at different cross-sectional positions for a target part of a subject based on a reflection signal of an ultrasonic wave output from a probe; measuring a measurement value related to the target part for each of the plurality of generated cross-sectional images; extracting a target measurement value as a measurement target based on a measurement result of a plurality of the measurement values; and extracting a target cross-sectional image corresponding to a target cross-sectional position based on evaluation results of the plurality of cross-sectional images.

### Advantageous Effects of Invention

According to the present invention, it is possible to extract an optimal measurement value and an optimal cross-sectional image.

### BRIEF DESCRIPTION OF THE DRAWINGS

FIG. 1 is a block diagram illustrating an example of an ultrasonic diagnostic apparatus according to an embodiment of the present invention;

FIG. 2 is a diagram provided for describing an example of the measurement of measurement values by a measurer in the ultrasonic diagnostic apparatus illustrated in FIG. 1;

FIG. 3 is a diagram provided for describing another example of the measurement of measurement values by the measurer in the ultrasonic diagnostic apparatus illustrated in FIG. 1;

FIG. 4 is a diagram provided for describing an example of target measurement value extraction by a target measurement value extractor in the ultrasonic diagnostic apparatus illustrated in FIG. 1;

FIG. 5 is a diagram provided for describing another example of the target measurement value extraction by the target measurement value extractor in the ultrasonic diagnostic apparatus illustrated in FIG. 1;

FIG. 6 is a flowchart provided for describing an example of a target result extraction method by the ultrasonic diagnostic apparatus illustrated in FIG. 1;

FIG. 7 is a diagram provided for describing the target result extraction method illustrated in FIG. 6;

FIG. 8 is a flowchart provided for describing another example (Variation 1) of the target result extraction method by the ultrasonic diagnostic apparatus illustrated in FIG. 1;

FIG. 9 is a flowchart provided for describing yet another example (Variation 2) of the target result extraction method by the ultrasonic diagnostic apparatus illustrated in FIG. 1; and

FIG. 10 is a flowchart provided for describing still another variation (Variation 3) of the target result extraction method by the ultrasonic diagnostic apparatus illustrated in FIG. 1.

### DESCRIPTION OF EMBODIMENTS

Hereinafter, an embodiment of the present invention will be described in detail with reference to the accompanying drawings.

### Ultrasonic Diagnostic Apparatus

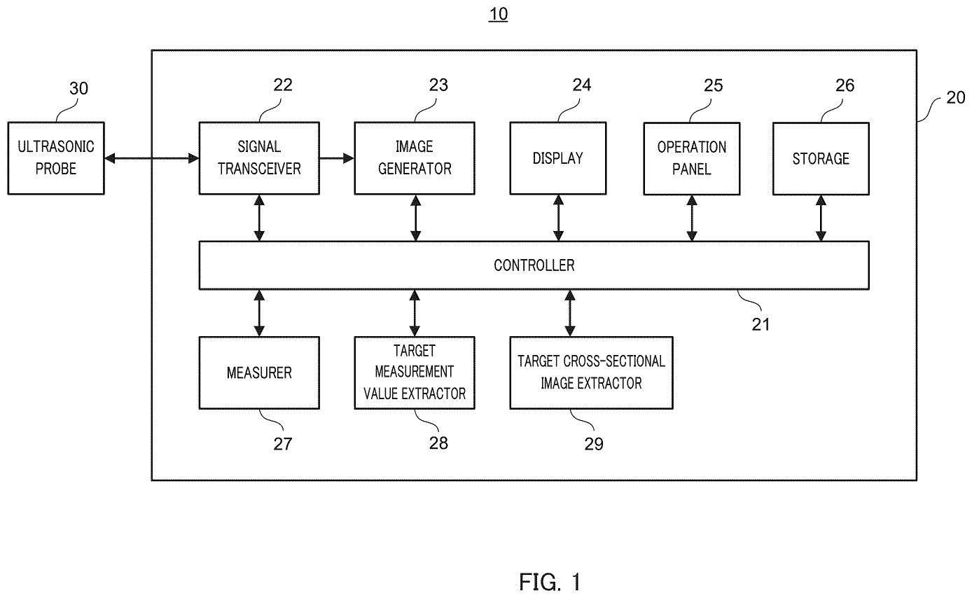

FIG. 1 is a block diagram illustrating an example of an ultrasonic diagnostic apparatus 10 according to the present embodiment.

The ultrasonic diagnostic apparatus 10 transmits and receives ultrasonic waves to and from a target part in a subject such as a living body, generates and displays, based on a reception signal obtained from a received ultrasonic echo, the shape, property, or dynamic state of the biological tissue of the target part as an ultrasonic image, and is used for a medical doctor or the like to perform image diagnosis.

As illustrated in FIG. 1, the ultrasonic diagnostic apparatus 10 includes an apparatus body 20 and an ultrasonic probe 30. In the ultrasonic diagnostic apparatus 10, the ultrasonic probe 30 is connected to the apparatus body 20 via a cable (illustration is omitted).

### Ultrasonic Probe

Although detailed illustration is omitted, the ultrasonic probe 30 includes a plurality of transducers formed of piezoelectric elements. For example, the plurality of transducers is arranged in a one-dimensional array in the scanning direction of ultrasonic waves. Note that, the transducers may also be arranged in a two-dimensional array.

In the ultrasonic probe 30, each transducer converts a driving signal output from a signal transceiver 22 of the apparatus body 20, which will be described later, into ultrasonic waves and transmits the ultrasonic waves into the subject, receives an ultrasonic echo, which is the ultrasonic waves reflected in the subject, and converts the ultrasonic echo into a reception signal, and outputs the reception signal to the signal transceiver 22.

As the ultrasonic probe 30, for example, various types of probes including a sector probe of a sector scanning method, a convex probe of a convex scanning method, a linear probe of a linear scanning method, an intracavitary probe, and the like can be used.

Note that, the connection between the apparatus body 20 and the ultrasonic probe 30 may be performed by wireless communication such as ultra wide band (UWB) instead of wired communication by a cable.

### Apparatus Body

As illustrated in FIG. 1, the apparatus body 20 includes a controller 21, a signal transceiver 22, an image generator 23, a display 24, an operation panel 25, and a storage 26. In addition, in the present embodiment, the apparatus body 20 includes a measurer 27, a target measurement value extractor 28, and a target cross-sectional image extractor 29.

The controller 21 is a so-called computer, and includes a central processing unit (CPU) as an arithmetic/control apparatus, a read only memory (ROM) and a random access memory (RAM) as main storage apparatuses, and the like.

In the controller 21, the ROM non-temporarily stores a program and setting data, and the CPU reads a program corresponding to the processing content from the ROM, develops the program in the RAM that temporarily stores the program, and executes the developed program. Thus, the controller 21 centrally controls the operation of each function block of the ultrasonic diagnostic apparatus 10. That is, the controller 21 controls the signal transceiver 22, the image generator 23, the display 24, the operation panel 25, the storage 26, the measurer 27, the target measurement value extractor 28, and the target cross-sectional image extractor 29 according to the respective functions thereof, thereby performing overall control of the ultrasonic diagnostic apparatus 10.

The signal transceiver 22 includes a transmitter and a receiver (illustration is omitted). Under the control of the controller 21, the transmitter supplies a driving signal, which is an electrical signal, to the ultrasonic probe 30 to cause the ultrasonic probe 30 to generate ultrasonic waves.

Although detailed illustration is omitted, the transmitter includes, for example, a clock generation circuit, a delay circuit, and a pulse generation circuit. The clock generation circuit is a circuit that generates a clock signal that determines transmission timing and a transmission frequency of the driving signal. The delay circuit is a circuit for setting a delay time for each individual path corresponding to each transducer of the ultrasonic probe 30, delaying transmission of a driving signal by the set delay time, and performing focusing (transmission beam forming) of a transmission beam formed by transmission ultrasonic waves. The pulse generation circuit is a circuit for generating a pulse signal as a driving signal at a set voltage and a set time interval.

Under the control of the controller 21, the transmitter configured as described above sequentially switches between the plurality of transducers to which the driving signal is supplied while shifting a predetermined number of the transducers for each transmission and reception of ultrasonic waves, and supplies the driving signal to the plurality of transducers for which the output is selected, thereby performing scanning.

In the signal transceiver 22, the receiver receives a reception signal, which is an electrical signal from the ultrasonic probe 30, according to the control of the controller 21.

Although detailed illustration is omitted, the receiver includes, for example, an amplifier, an A/D conversion circuit, and a phasing addition circuit. The amplifier is a circuit for amplifying a reception signal at a preset amplification factor for each individual path corresponding to each transducer. The A/D conversion circuit is a circuit for performing analog-to-digital conversion (A/D conversion) on the amplified reception signal. The phasing addition circuit is a circuit for giving a delay time to the A/D converted reception signal for each individual path corresponding to each transducer to adjust the time phase, and adding (phasing addition) those described above to generate sound ray data. That is, the phasing addition circuit generates sound ray data by performing reception beam forming on the reception signal for each transducer.

The image generator 23 generates cross-sectional images at different cross-sectional positions for a target part of a subject based on sound ray data that is a reflection signal of ultrasonic waves from the ultrasonic probe 30.

Specifically, under the control of the controller 21, the image generator 23 performs envelope detection processing, logarithmic compression, and the like on the sound ray data from the receiver of the signal transceiver 22, and performs luminance conversion by adjusting the dynamic range and the gain. Thus, the image generator 23 generates a B-mode image (hereinafter referred to as a cross-sectional image) as a two-dimensional cross-sectional image. That is, the cross-sectional image represents the intensity of the reception signal by luminance. Note that, the image generator 23 may also generate an A-mode image, an M-mode image (two-dimensional cross-sectional image), a Doppler image, a color mode image, or a three-dimensional image.

In addition, the image generator 23 includes an image memory (not illustrated) constituted by a semiconductor memory such as a dynamic random access memory (DRAM). The image generator 23 causes the image memory to store the generated cross-sectional images on a frame-by-frame basis.

In addition, the image generator 23 performs image processing such as image filter processing and time smoothing processing on a cross-sectional image read from the image memory, and converts the cross-sectional image into a display image pattern that is displayed on the display 24.

The display 24 is a display apparatus such as a liquid crystal display (LCD), a cathode-ray tube (CRT) display, an organic electronic luminescence (EL) display, an inorganic EL display, or a plasma display. Under the control of the controller 21, the display 24 displays a cross-sectional image generated by the image generator 23 on the display screen. In addition, in a case where a target measurement value and a target cross-sectional image, which will be described later, are extracted, the display 24 displays the target measurement value and the target cross-sectional image.

The operation panel 25 includes various switches, buttons, a track pad, a track ball, a mouse, a keyboard, a touch screen, and the like.

The operation panel 25 performs, for example, an input such as the activation, setting, selection, operation, and termination of an apparatus or an application. In addition, the operation panel 25 performs an input of data such as information of the subject, various parameters for displaying a cross-sectional image on the display 24, and the like. The operation panel 25 outputs a signal corresponding to an input to the controller 21.

The storage 26 is a storage apparatus capable of writing and reading information, such as a flash memory, a hard disk drive (HDD), or a solid state drive (SSD). The storage 26 stores, for example, a cross-sectional image generated by the image generator 23.

The measurer 27 performs the measurement of a set measurement item(s) on a cross-sectional image of a target part of a subject, which has been generated by the image generator 23. The measurement items are, for example, length, area, volume, or the like obtained by morphological measurement using a cross-sectional image. In a case where the target part of the subject is a fetus of a pregnant woman, examples of the measurement items related to the fetus include the CRL, bi-parietal diameter (hereinafter referred to as BPD), abdominal circumference (hereinafter referred to as AC), femur length (hereinafter referred to as FL), and the like. The controller 21 causes measurement items such as the CRL, BPD, AC, and FL to be displayed on the display 24, and the user (for example, a medical doctor) selects and sets a desired measurement item by using the operation panel 25.

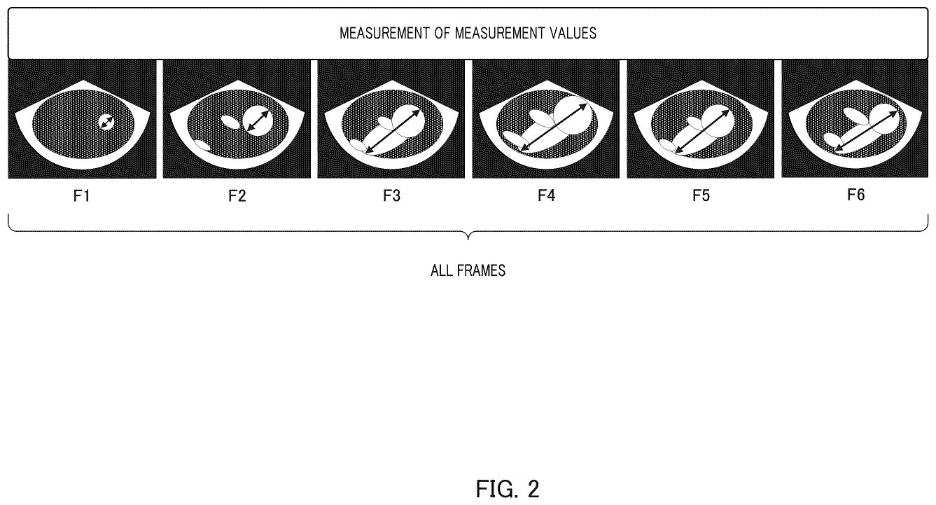

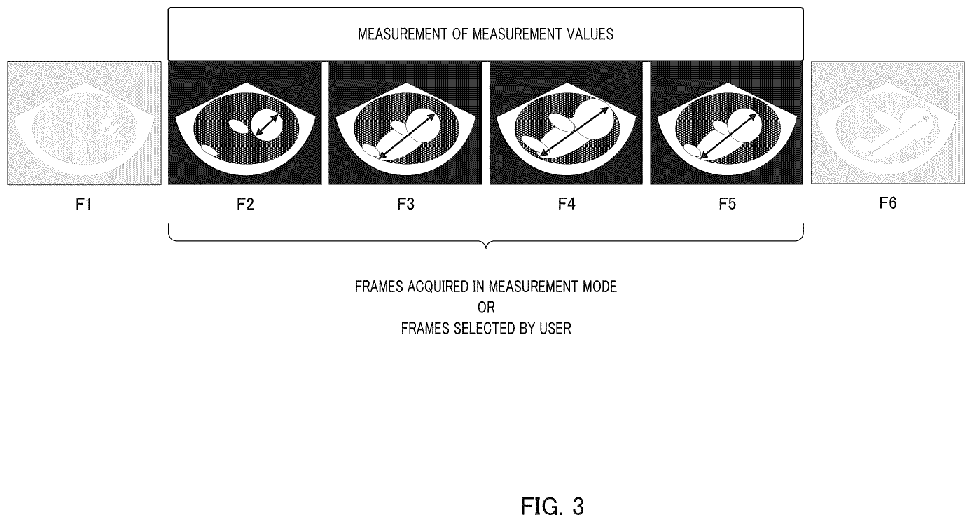

The measurer 27 will be described with reference to FIGS. 2 and 3. FIG. 2 is a diagram provided for describing an example of the measurement of measurement values by the measurer 27 in the ultrasonic diagnostic apparatus 10. FIG. 3 is a diagram provided for describing another example of the measurement of measurement values by the measurer 27 in the ultrasonic diagnostic apparatus 10.

As illustrated in FIG. 2, the measurer 27 performs the measurement of a set measurement item(s) on all cross-sectional images (here, as an example, all frames F1 to F6) generated by the image generator 23. For example, when a cross-sectional image is generated by the image generator 23 while the target part of the subject is being scanned with the ultrasonic probe 30, the measurement on all frames is performed by performing the measurement of the set measurement item(s) each time the cross-sectional image is generated.

In addition, the target part of the subject is scanned with the ultrasonic probe 30, and all the cross-sectional images generated by the image generator 23 are stored in the storage 26. Then, after the scanning ends, the measurement on all the frames may be performed by performing the measurement of the set measurement item(s) on all the cross-sectional images stored (saved) in the storage 26 by the scanning. In this case, the target measurement value extractor 28 and the target cross-sectional image extractor 29, which will be described later, are operated after the completion of the measurement.

In addition, as illustrated in FIG. 3, the measurer 27 may perform the measurement of a set measurement item(s) on more than one cross-sectional image (here, as an example, frames F2 to F5 in all frames F1 to F6) of cross-sectional images generated by the image generator 23. For example, it is assumed that there is a measurement mode (for example, a CRL measurement mode or the like) for a particular measurement item as a function of the ultrasonic diagnostic apparatus 10. In this case, during the period in which the measurement mode is selected, the measurement of the set measurement item is performed on the cross-sectional image(s) (frame(s)) generated by the image generator 23. In addition, a measurement period (the start time and the end time) for measuring a measurement item may be specified by a user operation, and even in such a case, the measurement of the set measurement item is performed on the cross-sectional image(s) (frame(s)) generated by the image generator 23 during the specified measurement period.

Here, for example, in a case where the CRL is measured, a fetus which is a target part may not appear in a cross-sectional image. Even in this case, the measurer 27 performs the measurement and sets the measurement value to 0. In addition, a fetus which is a target part may only partially appear in a cross-sectional image (for example, frames F1 and F2 in FIG. 2). Even in this case, the measurer 27 performs the measurement to measure the measurement value of the portion estimated to be the target part. The measurement value in this case is smaller than the measurement value in the cross-sectional image on the median sagittal plane which is the appropriate cross-sectional position.

Note that, the measurer 27 is configured to recognize, for example, by using a learning processor and a learning model, which will be described later, the target part to be measured.

The target measurement value extractor 28 extracts a target measurement value as a measurement target based on a measurement result of a plurality of measurement values measured by the measurer 27.

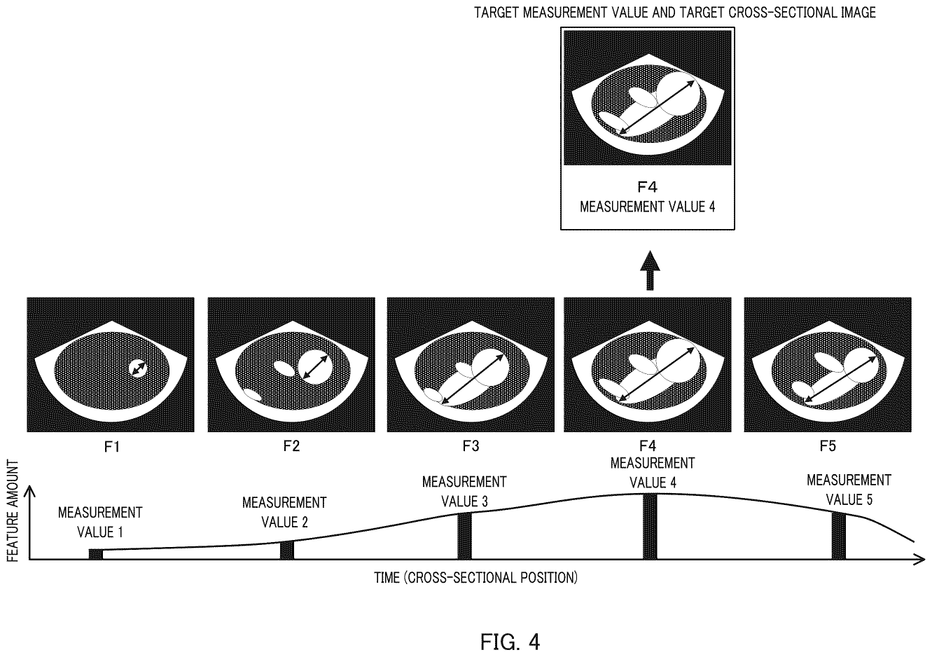

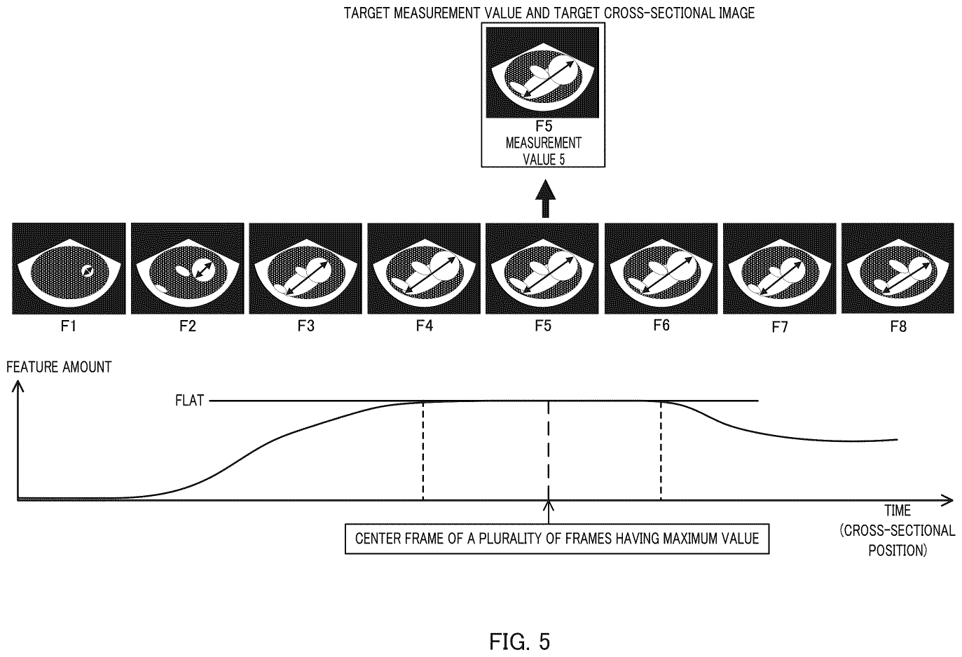

The target measurement value extractor 28 will be described with reference to FIGS. 4 and 5. FIG. 4 is a diagram provided for describing an example of target measurement value extraction by the target measurement value extractor 28 in the ultrasonic diagnostic apparatus. In addition, FIG. 5 is a diagram provided for describing another example of the target measurement value extraction by the target measurement value extractor 28 in the ultrasonic diagnostic apparatus 10.

For the plurality of measurement values measured by the measurer 27, the target measurement value extractor 28 extracts the target measurement value based on a tendency (the measurement result) of a change in the plurality of measurement values with respect to the cross-sectional positions.

For example, in a case where the CRL is measured, as illustrated in FIG. 4, the measurement value in the cross-sectional image (the frame F4) on the median sagittal plane of the fetus is the largest, and the measurement values in the other cross-sectional images (the frames F1 to F3 and F5) are small. Accordingly, in this case, the target measurement value is extracted based on the maximum value (the measurement result) among the plurality of measurement values. At this time, the frame F4 having the maximum value may be set as a target cross-sectional image (or a target cross-sectional image candidate) to be described later.

Depending on the measurement item, the target measurement value may be extracted based on the minimum value among the plurality of measurement values. In addition, as exemplified in FIG. 4, it is ideal that the change in the measurement value with respect to the cross-sectional position has one maximum value (or minimum value). However, some measurement items may have a local maximum value or a local minimum value. In such a case, the target measurement value may be extracted based on the local maximum value or the local minimum value among the plurality of measurement values. As described above, the target measurement value is extracted based on an extremum (the measurement result) among the plurality of measurement values.

In addition, as illustrated in FIG. 5, there is also a case where the plurality of measurement values includes the maximum value that occurs a plurality of times, and in such a case, the measurement value in the frame F5 in the center of the frames F4 to F6 having the maximum value is set as the target measurement value. At this time, the frame F5 in the center may be set as a target cross-sectional image (or a target cross-sectional image candidate) to be described later.

The target cross-sectional image extractor 29 extracts a target cross-sectional image corresponding to a target cross-sectional position based on evaluation results of a plurality of cross-sectional images generated by the image generator 23.

For example, in a case where the CRL is measured, the cross-sectional position on the median sagittal plane of the fetus is the target cross-sectional position, and thus, the target cross-sectional image corresponding to the position is evaluated and extracted using, for example, scores to be described later.

The target cross-sectional image extractor 29 includes, for example, a learning processor and a learning model (illustration is omitted) in order to obtain scores for extracting the target cross-sectional image.

The learning processor generates the learning model by, for example, deep learning using training data. The training data is, for example, a cross-sectional image of a fetus including a median sagittal plane, or the like, and the learning model is optimized such that appropriate outputs are obtained for various inputs by repeatedly performing supervised learning using a large amount of various training data.

The learning model is mainly formed of, for example, a neural network. The learning model outputs each score of an identified target part based on a plurality of cross-sectional images generated by the image generator 23. The score indicates the appropriateness of an identified target part, and a target cross-sectional image having a high score (high appropriateness) is extracted based on scores in a plurality of cross-sectional images.

In this way, the target measurement value extractor 28 and the target cross-sectional image extractor 29 extract the target measurement value and the target cross-sectional image, respectively. Then, when it is determined that the target measurement value and the target cross-sectional image are optimal by the determination to be described later, the target measurement value and the target cross-sectional image determined as optimal are displayed on the display 24 and presented to the user.

The signal transceiver 22, the image generator 23, the measurer 27, the target measurement value extractor 28, and the target cross-sectional image extractor 29 as described above are constituted by, for example, dedicated or general-purpose hardware (electronic circuit) corresponding to each processing. Note that, the signal transceiver 22, the image generator 23, the measurer 27, the target measurement value extractor 28, and the target cross-sectional image extractor 29 may be configured such that processing in each unit is executed by a CPU or a graphics processing unit (GPU). In this case, the CPU or the GPU includes a program for performing processing in each unit.

### Target Result Extraction Method

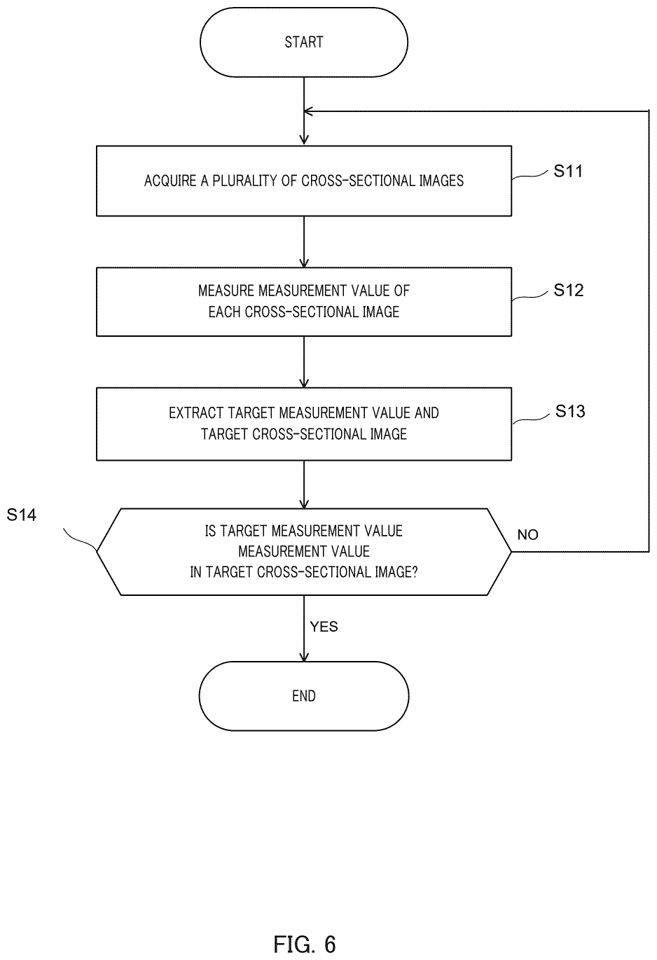

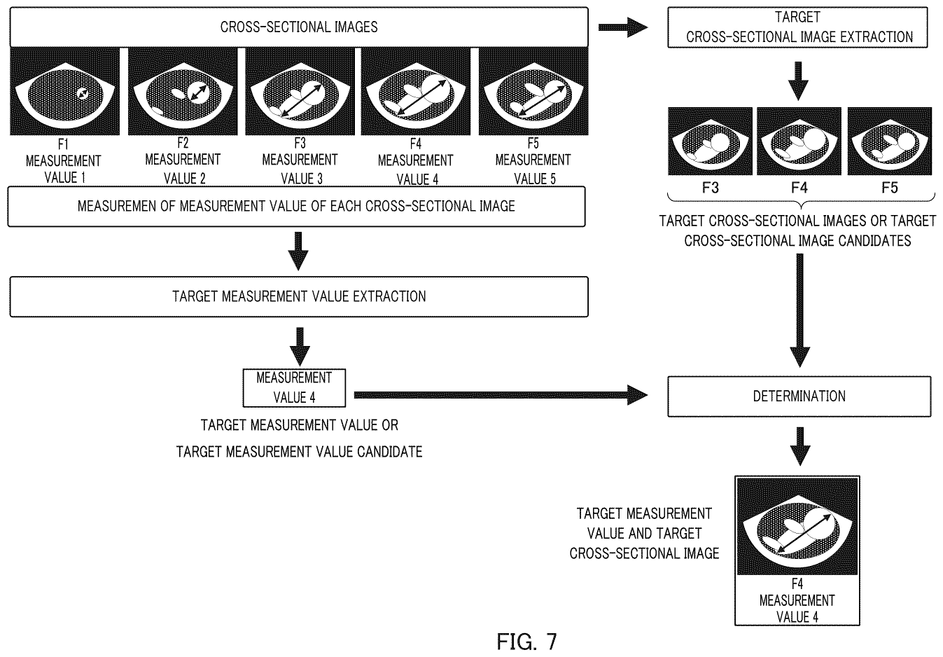

FIG. 6 is a flowchart provided for describing an example of a target result extraction method by the ultrasonic diagnostic apparatus 10. FIG. 7 is a diagram provided for describing the target result extraction method illustrated in FIG. 6. The target result extraction method by the ultrasonic diagnostic apparatus 10 is performed by the controller 21 executing a target result extraction program. Note that, hereinafter, as an example, a case where CRL measurement is performed will be described.

### (Step S11)

The user (for example, a medical doctor) applies the ultrasonic probe 30 of the ultrasonic diagnostic apparatus 10 to the abdomen or the like of a pregnant woman as a subject and scans a region in which a fetus is present, which is a target part, whereby the controller 21 acquires a plurality of cross-sectional images by the signal transceiver 22 and the image generator 23. As an example, as illustrated in FIG. 7, it is assumed that the controller 21 acquires cross-sectional images of the frames F1 to F5.

### (Step S12)

The controller 21 causes the measurer 27 to measure the measurement value of each cross-sectional image. Here, as described above, the CRL is measured, and for example, when a cross-sectional image is generated during scanning with the ultrasonic probe 30, the CRL measurement is performed every time the cross-sectional image is generated, and thus, the measurement is performed on all the frames F1 to F5.

### (Step S13)

The controller 21 causes the target measurement value extractor 28 and the target cross-sectional image extractor 29 to extract the target measurement value and the target cross-sectional image. Here, the target measurement value extractor 28 and the target cross-sectional image extractor 29 are operated in parallel (at the same time), and extract the operation measurement value and the target cross-sectional image, respectively. Note that, in the present embodiment, unlike in PTL 1 described above, the target cross-sectional image extractor may be operated after the target measurement value extractor is operated.

For example, as described with reference to FIG. 4, the target measurement value extractor 28 extracts, based on the maximum value among the plurality of measurement values, a measurement value, which is the maximum value, as the target measurement value. In addition, as described above, the target cross-sectional image extractor 29 extracts a target cross-sectional image having a high score based on scores in the plurality of cross-sectional images.

### (Step S14)

The controller 21 determines whether the target measurement value is the measurement value in the target cross-sectional image. In a case where the target measurement value is the measurement value in the target cross-sectional image (YES), a series of flows is ended, whereas in a case where the target measurement value is not the measurement value in the target cross-sectional image (NO), the process returns to step S11.

Since the target measurement value extracted by the target measurement value extractor 28 is the measurement value as the maximum value among a plurality of measurement values, it can be determined as the optimal target measurement value in the case of the measurement of the CRL. In addition, in a case where the target measurement value is the measurement value in the target cross-sectional image, it can be determined that the target cross-sectional image is also optimal. Accordingly, in a case where the target measurement value is the measurement value in the target cross-sectional image, the controller 21 causes the display 24 to display the target measurement value and the target cross-sectional image, which have been determined to be optimal, and causes the target measurement value and the target cross-sectional image to be presented to the user. In this way, the target measurement value and the target cross-sectional image, which have been determined to be optimal, are displayed on the display 24 and presented to the user, and thus, the accuracy of measurement at the time of diagnosis improves and the efficiency also improves.

For example, as illustrated in FIG. 7, it is assumed that a target measurement value extracted by the target measurement value extractor 28 is the measurement value 4 in the frame F4, and target cross-sectional images (target cross-sectional image candidates) extracted by the target cross-sectional image extractor 29 are the frames F3 to F5. In this case, since the measurement value 4 in the frame F4 is the target measurement value among the measurement values 3 to 5 in the frames F3 to F5, the controller 21 determines the cross-sectional image in the frame F4 as the optimal target cross-sectional image.

On the other hand, in a case where the target measurement value is not the measurement value in the target cross-sectional image, there may be a problem that the arrangement position of the ultrasonic probe 30 is bad or the like. For this reason, for example, the controller 21 causes the display 24 to display a message such as “Please check the arrangement position of the ultrasonic probe 30” to prompting the user to perform remeasurement.

As described above, the ultrasonic diagnostic apparatus 10 of the present embodiment includes a target measurement value extractor and a target cross-sectional image extractor. The target measurement value extractor extracts a target measurement value as a measurement target based on the measurement result of a plurality of measurement values. The target cross-sectional image extractor extracts a target cross-sectional image corresponding to a target cross-sectional position based on evaluation results of the plurality of cross-sectional images.

According to the ultrasonic diagnostic apparatus 10 of the present embodiment configured as described above, the target measurement value as the measurement target is extracted based on the measurement result of a plurality of measurement values, for example, based on a tendency of a change in the plurality of measurement values or based on an extremum among the plurality of measurement values, and thus, it is possible to extract the optimal measurement value. In addition, the target cross-sectional image corresponding to the target cross-sectional position can be extracted based on the evaluation results of the plurality of cross-sectional images, for example, based on the above-described scores. That is, only by scanning a target part (for example, a fetus) with the ultrasonic probe 30, the user can automatically obtain an optimal target measurement value and an optimal target cross-sectional image without performing any other operation, for example, freezing a cross-sectional image or setting a measurement position. As a result, the accuracy of measurement at the time of diagnosis can be improved, and the efficiency can also be improved.

### <Variation 1>

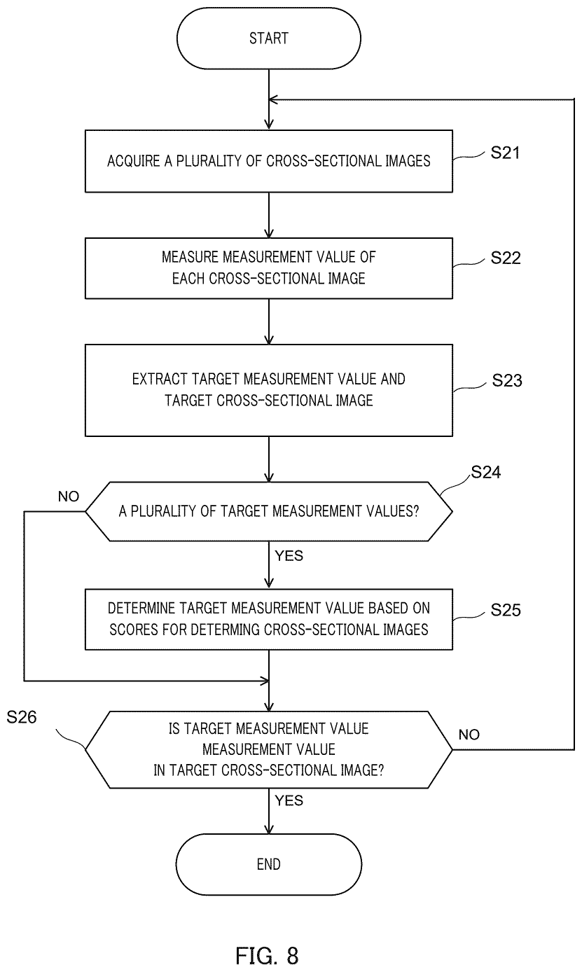

FIG. 8 is a flowchart provided for describing another example of the target result extraction method by the ultrasonic diagnostic apparatus 10.

In the present variation, steps S21 to S23 and step S26 are the same as steps S11 to S13 and step S14 in the flowchart illustrated in FIG. 6. Accordingly, differences from those in the flowchart illustrated in FIG. 6 will be mainly described here.

### (Step S24)

The controller 21 checks whether a plurality of the measurement values has been extracted by the target measurement value extractor 28. In the case of a plurality of the target measurement values (YES), the processing proceeds to step S25, whereas in the case of not a plurality of the target measurement values (NO), the processing proceeds to step S26.

### (Step S25)

In the case of a plurality of the target measurement values (a plurality of target measurement value candidates), the controller 21 determines, based on the scores in the plurality of cross-sectional images, the target measurement value in the target cross-sectional image having a high score as the optimal target measurement value.

In this way, even in the case of a plurality of the target measurement values, the optimal target measurement value can be determined based on the scores in the plurality of cross-sectional images.

### <Variation 2>

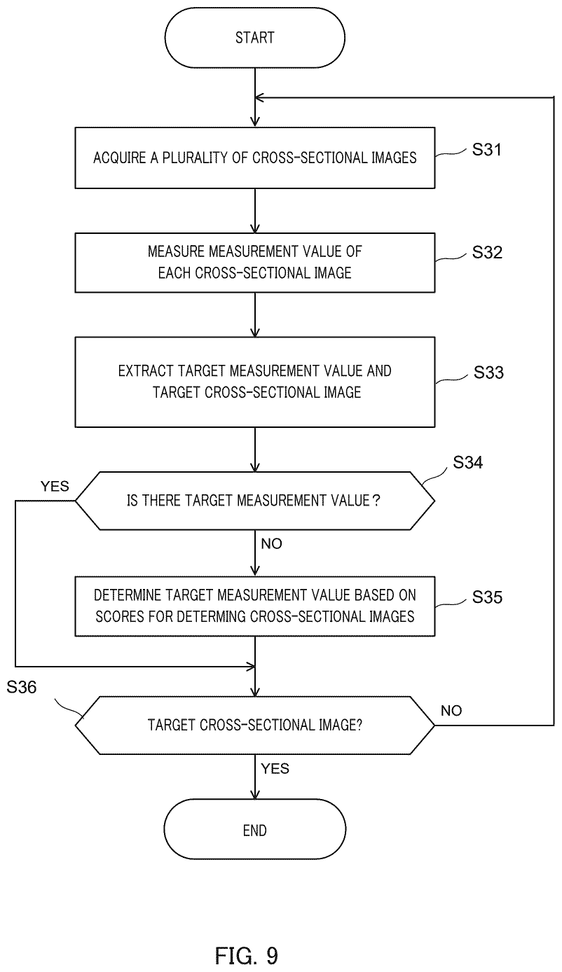

FIG. 9 is a flowchart provided for describing yet another example of the target result extraction method by the ultrasonic diagnostic apparatus 10.

Even in the present variation, steps S31 to S33 and step S36 are the same as steps S11 to S13 and step S14 in the flowchart illustrated in FIG. 6. Accordingly, differences from those in the flowchart illustrated in FIG. 6 will be mainly described here as well.

### (Step S34)

The controller 21 checks whether there is a target measurement value extracted by the target measurement value extractor 28. In a case where there is a target measurement value (YES), the processing proceeds to step S36, whereas in a case where there is no target measurement value (NO), the processing proceeds to step S35.

### (Step S35)

In a case where there is no measurement value, the controller 21 determines, based on the scores in the plurality of cross-sectional images, the measurement value of the target cross-sectional image having a high score as the optimal target measurement value.

As described above, even in a case where no target measurement value can be extracted, the optimal target measurement value can be determined based on the scores in the plurality of cross-sectional images.

### <Variation 3>

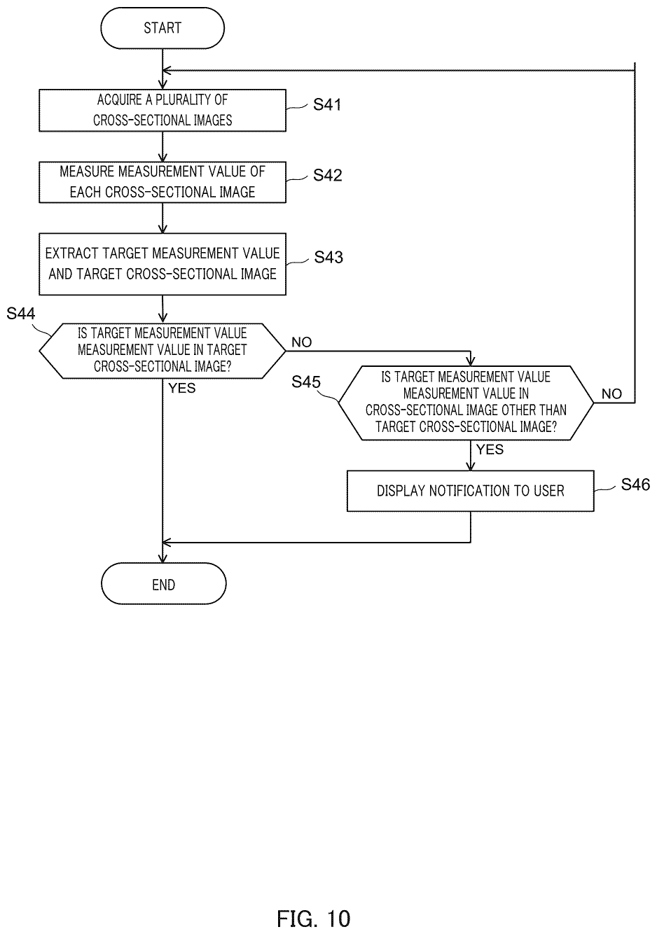

FIG. 10 is a flowchart provided for describing still another example of the target result extraction method by t the ultrasonic diagnostic apparatus 10.

In the present variation, steps S41 to S44 are the same as steps S11 to S14 in the flowchart illustrated in FIG. 6. Accordingly, differences from those in the flowchart illustrated in FIG. 6 will be mainly described here as well.

### (Step S45)

The controller 21 checks whether the target measurement value extracted by the target measurement value extractor 28 is a measurement value in a cross-sectional image other than the target cross-sectional image extracted by the target cross-sectional image extractor 29. In a case where the target measurement value is a measurement value in a cross-sectional image other than the target cross-sectional image (YES), the processing proceeds to step S46, whereas in a case where the target measurement value is not a measurement value in a cross-sectional image other than the target cross-sectional image (NO), the processing returns to step S41.

### (Step S46)

In a case where the target measurement value is a measurement value in a cross-sectional image other than the target cross-sectional image, the controller 21 notifies the user of the case, causes the display 24 to display the target measurement value and the measurement value for requesting the user for confirmation, and ends the processing. In this case, the above-described score may also be displayed on the display 24.

As described above, in a case where the measurement value is a measurement value in a cross-sectional image other than the target cross-sectional image, information necessary for the user's determination is displayed on the display 24 to request the user for confirmation.

Any of the embodiment and variations described above is only illustration of an exemplary embodiment for implementing the present invention, and the technical scope of the present invention shall not be construed limitedly thereby. That is, the present invention can be implemented in various forms without departing from the spirit or main features thereof.

The disclosure of Japanese Patent Application No. 2023-095504, filed on Jun. 9, 2023, including the specification, drawings and abstract, is incorporated herein by reference in its entirety.

### REFERENCE SIGNS LIST

10 Ultrasonic diagnostic apparatus
20 Apparatus body
21 Controller
22 Signal transceiver
23 Image generator
24 Display
25 Operation panel
26 Storage
27 Measurer
28 Target measurement value extractor
29 Target cross-sectional image extractor
30 Ultrasonic probe

1. An ultrasonic diagnostic apparatus, comprising:
an image generator that generates a plurality of cross-sectional images at cross-sectional positions for a target part of a subject based on a reflection signal of an ultrasonic wave output from a probe, the cross-sectional positions being different cross-sectional positions;
a measurer that measures a measurement value related to the target part for each of the plurality of generated cross-sectional images;
a target measurement value extractor that extracts a target measurement value as a measurement target based on a measurement result of a plurality of the measurement values; and
a target cross-sectional image extractor that extracts a target cross-sectional image corresponding to a target cross-sectional position based on evaluation results of the plurality of cross-sectional images.

2. The ultrasonic diagnostic apparatus according to claim 1, comprising a display that displays the extracted target measurement value and the extracted target cross-sectional image.

3. The ultrasonic diagnostic apparatus according to claim 1, which has a measurement mode for measuring the measurement value, wherein
in the measurement mode,
the image generator generates the plurality of cross-sectional images, and
the measurer measures the measurement value for each of the plurality of cross-sectional images generated by the image generator, respectively.

4. The ultrasonic diagnostic apparatus according to claim 1, wherein
the measurer measures the measurement value for, among the plurality of generated cross-sectional images, each of more than one cross-sectional image specified by a user.

5. The ultrasonic diagnostic apparatus according to claim 1, wherein:
the measurement result is an extremum among the plurality of measurement values, and
the target measurement value extractor extracts the target measurement value based on the extremum.

6. The ultrasonic diagnostic apparatus according to claim 1, wherein:
the measurement result is a tendency of a change in a plurality of the measurement values with respect to the cross-sectional positions, and
the target measurement value extractor extracts the target measurement value based on the tendency.

7. The ultrasonic diagnostic apparatus according to claim 1, wherein:
the evaluation results are scores for evaluating appropriateness of the target part in the plurality of cross-sectional images,
the target cross-sectional image extractor extracts the target cross-sectional image based on the scores, and
in a case where a plurality of the target measurement values is extracted, the target measurement value extractor extracts the target measurement value from the plurality of extracted measurement values based on the scores.

8. The ultrasonic diagnostic apparatus according to claim 1, wherein:
the evaluation results are scores for evaluating appropriateness of the target part in the plurality of cross-sectional images,
the target cross-sectional image extractor extracts the target cross-sectional image based on the scores, and
in a case where the target measurement value is not extracted, the target measurement value extractor extracts, as the measurement value, a measurement value in the target cross-sectional image extracted based on the scores.

9. The ultrasonic diagnostic apparatus according to claim 1, wherein
in a case where the extracted target measurement value is a measurement value in the target cross-sectional image extracted by the target cross-sectional image extractor, the target measurement value extractor determines that an optimal target measurement value has been extracted.

10. The ultrasonic diagnostic apparatus according to claim 2, wherein
in a case where the extracted target measurement value is not a measurement value in the target cross-sectional image extracted by the target cross-sectional image extractor, the display notifies a user of the case and displays the target measurement value and the measurement value for requesting the user for confirmation.

11. The ultrasonic diagnostic apparatus according to claim 1, wherein:
in a case where scanning of the target part is being performed with the probe, the measurer measures the measurement value when the cross-sectional image is generated, and
the target measurement value extractor extracts the target measurement value based on the measurement result after the scanning ends.

12. The ultrasonic diagnostic apparatus according to claim 1, wherein:
the measurer performs measurement of the measurement value for each of the plurality of cross-sectional images that has been saved, and
the target measurement value extractor extracts the target measurement value based on the measurement result after the measurement ends.

13. The ultrasonic diagnostic apparatus according to claim 1, wherein
the target measurement value extractor and the target cross-sectional image extractor are operated in parallel.

14. The ultrasonic diagnostic apparatus according to claim 1, wherein
the target cross-sectional image extractor is operated after the target measurement value extractor is operated.

15. A target result extraction method, comprising:
generating a plurality of cross-sectional images at different cross-sectional positions for a target part of a subject based on a reflection signal of an ultrasonic wave output from a probe;
measuring a measurement value related to the target part for each of the plurality of generated cross-sectional images;
extracting a target measurement value as a measurement target based on a measurement result of a plurality of the measurement values; and
extracting a target cross-sectional image corresponding to a target cross-sectional position based on evaluation results of the plurality of cross-sectional images.

16. A non-transitory computer-readable recording medium storing a target result extraction program that causes a computer to execute:
generating a plurality of cross-sectional images at different cross-sectional positions for a target part of a subject based on a reflection signal of an ultrasonic wave output from a probe;
measuring a measurement value related to the target part for each of the plurality of generated cross-sectional images;
extracting a target measurement value as a measurement target based on a measurement result of a plurality of the measurement values; and
extracting a target cross-sectional image corresponding to a target cross-sectional position based on evaluation results of the plurality of cross-sectional images.
